(19) United States
(12) Patent Application Publication
(10) Pub. No.: US 2014/0171750 A1
(43) Pub. Date: Jun. 19, 2014

(54) ELECTRONIC INTUBATION

(71) Applicants: Aaron Fay, Brookline, MA (US); Evan Foss, Holliston, MA (US)

(72) Inventors: Aaron Fay, Brookline, MA (US); Evan Foss, Holliston, MA (US)

(73) Assignee: MASSACHUSETTS EYE & EAR INFIRMARY, Boston, MA (US)

(21) Appl. No.: 14/114,473

(22) PCT Filed: Apr. 30, 2012
(86) PCT No.: PCT/US12/35895

§ 371 Date: Feb. 10, 2014

Related U.S. Application Data
(60) Provisional application No. 61/481,126, filed on Apr. 29, 2011.

(51) Int. Cl. A61B 5/06; A61M 27/00

(52) U.S. Cl. CPC A61B 5/061; A61M 27/002
USPC 600/300; 604/8

(57) ABSTRACT

The disclosure relates to systems and methods for electronic intubation of bodily passages such as the nasolacrimal duct. The disclosed technology can be embodied in an apparatus that includes an electrically conductive probe configured to be extendible through a body passage, and a conductive tool for locating the probe in the body passage. The apparatus also includes a signal generator configured to generate a feedback signal, and a circuit coupled to the probe and the conductive tool. The circuit includes a comparison module that is configured to compare a voltage developed at the probe with a reference voltage and cause a generation of the feedback signal if at least a portion of the conductive tool touches the probe. The circuit is further configured to prevent the generation of the feedback signal when no portion of the conductive tool touches the probe.

### RELATED APPLICATION

This application claims priority to U.S. Provisional Application No. 61/481,126, filed Apr. 29, 2011, the entire content of which is incorporated herein by reference.

### TECHNICAL FIELD

The present disclosure relates generally to the field of medicine and, in particular, to new and useful methods and apparatus for enhanced intubation of the lacrimal ducts and the like.

### BACKGROUND

Lacrimal fluids or “tears” are normally supplied continuously to the eye socket from the lacrimal gland. Normally, excess fluid is drained through canaliculi or small passageways commencing adjacent the inner corner of the eye. The fluid is collected in the lacrimal sac and then drained via the nasolacrimal duct, which leads the fluid to the inferior nasal meatus.

If the lacrimal ducts become blocked, the fluid can no longer flow to the nasal meatus. Such closure can result from congenital anomalies, accidents, inflammation and advanced aging, as well as other physiological conditions. The result is that the eye is continually brimming over with tear fluid causing discomfort to the patient.

Nasolacrimal intubation has been a standard of care for severe epiphora, failed pediatric probings and irrigations, as well as an adjunct to dacriocystorhinostomy. The challenges of retrieving the metal portion of the silicone tubes as they descend in the posterior nasal cavity have resulted in various methods and devices. Because the procedures can cause bleeding of mucosa and turbinates, visibility and thus tube retrieval may be difficult. Many areas of the world still use a clamp to probe blindly in the nose to find the other end of the tube.

### SUMMARY

In one aspect, an apparatus includes an electrically conductive probe configured to be extendible through a body passage, and a conductive tool for locating the probe in the body passage. The apparatus also includes a signal generator configured to generate a feedback signal, and a circuit coupled to the probe and the conductive tool. The circuit includes a comparison module that is configured to compare a voltage developed at the probe with a reference voltage and cause a generation of the feedback signal if at least a portion of the conductive tool touches the probe. The circuit is further configured to prevent the generation of the feedback signal when no portion of the conductive tool touches the probe. The apparatus further includes a power source for powering the signaling mechanism to generate the signal, wherein the circuit is configured such that a current through the probe is below a first predetermined threshold.

In another aspect, an apparatus includes an electrically conductive probe configured to be extendible through a body passage a signal generator configured to generate a feedback signal, and a circuit coupled to the probe. The circuit includes a comparison module that is configured to compare a voltage developed at the probe with a reference voltage and cause a generation of the feedback signal if at least a portion of the probe touches a conductive tool used to locate the probe in the body passage. The circuit is further configured to prevent the generation of the feedback signal when no portion of the conductive tool touches the probe. The circuit is configured such that a current through the probe is below a first predetermined threshold.

In another aspect, an apparatus includes a signal generator configured to generate a feedback signal, and a circuit configured to be coupled to an electrically conductive probe that is extendible through a body passage and a conductive tool for locating the probe within the body passage. The circuit includes a comparison module that is configured to compare a voltage developed at the probe with a reference voltage and to cause a generation of the feedback signal if at least a portion of the probe touches the conductive tool. The circuit is further configured to prevent generation of the feedback signal when no portion of the conductive tool touches the probe. The circuit is further configured such that a current through the probe is below a first predetermined threshold.

In another aspect a method includes providing a circuit. The circuit is configured to be coupled to an electrically conductive probe that is extendible through a body passage, and a conductive tool for locating the probe within the body passage. The circuit includes a comparison module that is configured to compare a voltage developed at the probe with a reference voltage and cause a generation of a feedback signal. The circuit is further configured to prevent the generation of the feedback signal when no portion of the conductive tool touches the probe, and to limit the current through the probe to a value below a first predetermined threshold. The method also includes locating, based on the feedback signal, the probe when at least a portion of the probe touches the conductive tool.

Implementations of the apparatuses and method described above can include any combination of the following.

The signal generator can be selected from a group consisting of a visual signal generator, an audible signal generator, and a tactile signal generator. At least a portion of the circuit can be disposed on the conductive tool. At least a portion of the circuit can be disposed on a housing coupled to the probe. A response time of the comparison module can be greater than a second predetermined threshold. The second predetermined threshold can be 7 μs or between 2 μs and 7 μs. A shunt resistor can be connected in parallel to the probe and the conductive tool. The shunt resistor can be configured to limit current through the probe below the first predetermined threshold. The comparison module can include a comparator that is configured to withstand electrostatic discharge up to a third predetermined threshold value. Connectors can be provided to electrically couple the circuit with the probe and the conductive tool. The body passage can be a nasolacrimal duct. Any of the apparatuses above can be provided as a kit in a sterile package.

The apparatus and methods described herein can help, for example, where a nasal endoscope is not readily available, as an option to nasolacrimal surgeons. The combination of a feedback signal and proprioceptive sense of anatomy can facilitate efficient nasolacrimal intubation. The apparatus can include a circuit that limits the current (and resulting power dissipation) through the probe. Such limiting of the current in the probe allows for implementation of a continuity tester that can be safely used in human subjects without causing pain, discomfort or other adverse conditions that can result from high current flow through sensitive tissues. The methods and systems described herein allows for fast intubation through sensitive human body parts such as the lacrimal ducts.

Unless otherwise defined, all technical and scientific terms used herein have the same meaning as commonly understood by one of ordinary skill in the art to which this application belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a block diagram of an example of a system for facilitating intubation through a body passage

FIGS. 2 and 3A-3B each show a probe set and tool used on a patient.

FIGS. 4-6 are schematic diagrams of example circuits used in intubation systems.

### DETAILED DESCRIPTION

In nasolacrimal intubation procedures, retrieving the metal portion (or conducting probe portion) of a silicone tube as the probe descends in the posterior nasal cavity is challenging. This is due, at least in part, to the lack of visibility in the area where the probe is located and pulled out from using a tool such as a hook. One approach to overcome the challenges of locating the probe within a human or animal body is to use a conductive retrieval tool in conjunction with an electronic circuit that provides a feedback signal to the user when the tool and the probe are electrically coupled within a body passage. However, the use of such a circuit is challenging for various reasons. For example, because tissues within a body passage may conduct electricity, an electrical coupling between the probe and the conductive tool may be established if the probe and tool are sufficiently proximate to one another, but not actually touching. This can result in undesirable false positives and potential injuries to the tissues within the body passage if the user attempts to retrieve the probe when the tool is not actually touching the probe. Furthermore, if the current through the probe is above a certain level (for example, 200 μA), the current can burn the tissue as well as increase the chances of the false positives. On the other hand, implementing a system that is adequately sensitive and generates suitable feedback signals under such low current conditions is also challenging.

The systems and methods described herein can allow safe and efficient intubation through a body passage (e.g., nasolacrimal intubation) by reducing false positives and keeping a current through the probe below a predetermined threshold value that is safe yet still highly effective for use in human and animal subjects. This is made possible by new circuit designs that realize the above goals without compromising effectiveness or sensitivity of the overall systems.

The systems and methods described herein can be implemented in many ways, and some useful implementations are described below.

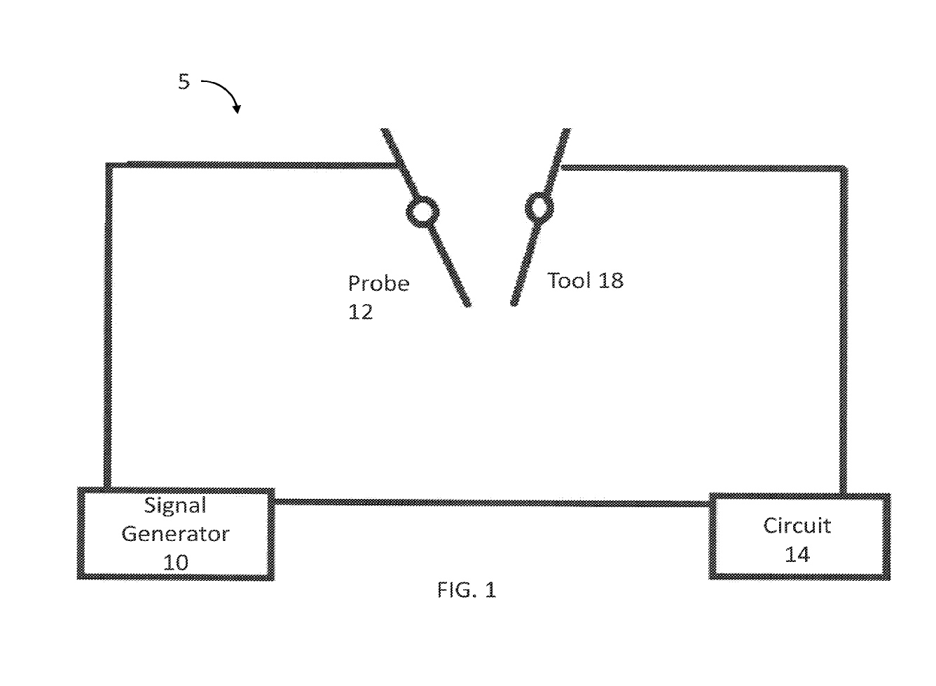

FIG. 1 is a block diagram of an example of a system 5 for facilitating intubation through a body passage. System 5 includes a probe 12 that can be inserted into a body passage such as a nasolacrimal duct. System 5 also includes a conductive tool 18 that is used to locate one end of probe 12 in the body passage, and locate and remove an end portion of the probe from the body passage. System 5 further includes a circuit 14 for detecting a physical contact between probe 12 and tool 18 within the body passage. Circuit 14 can be configured such the circuit prevents a current from passing through the probe 12 and the tool 18 unless they are in physical contact with one another. This reduces the possibility of false positives resulting from current passing from probe 12 to tool 18 through intervening body tissues when probe 12 and tool 18 are proximate to one another but not in actual physical contact. This can be done, for example, by connecting a shunt resistor in parallel across probe 12 and tool 18. When probe 12 and tool 18 are in contact with one another, circuit 14 can be configured to activate a signal generator 10 to generate a feedback signal.

Signal generator 10 can be an audio signal generator such as a buzzer. For example, a Sonalert® 1.5V, 3 mA SNP2 buzzer can be used. In some implementations, signal generator 10 can be a visual signal generator such as a light emitting diode (LED) or a display device such as a liquid crystal display (LCD) or LED screen. In some implementations, signal generator 10 can be a tactile signal generator such as a vibrator. Circuit 14 can be powered by, for example, a battery, e.g., a disposable or rechargeable battery. In some implementations, circuit 14 can include a powering module that includes a housing for a 1.5V battery.

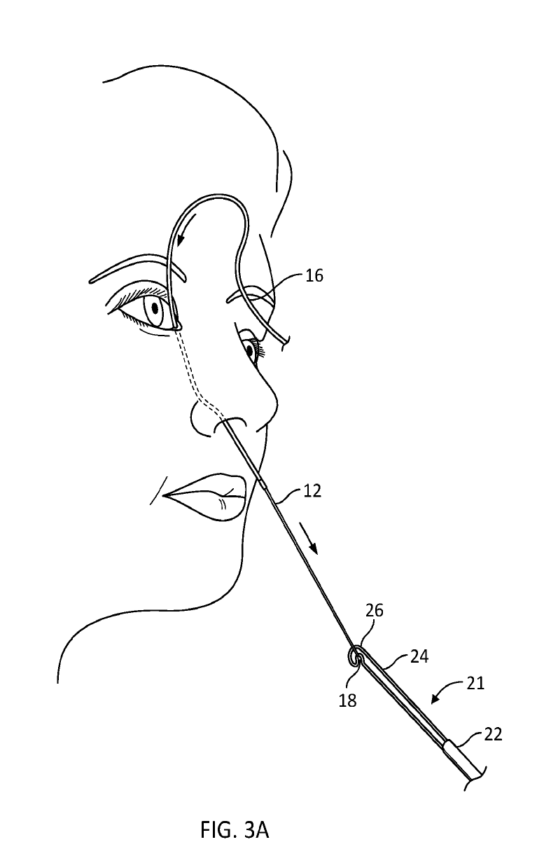

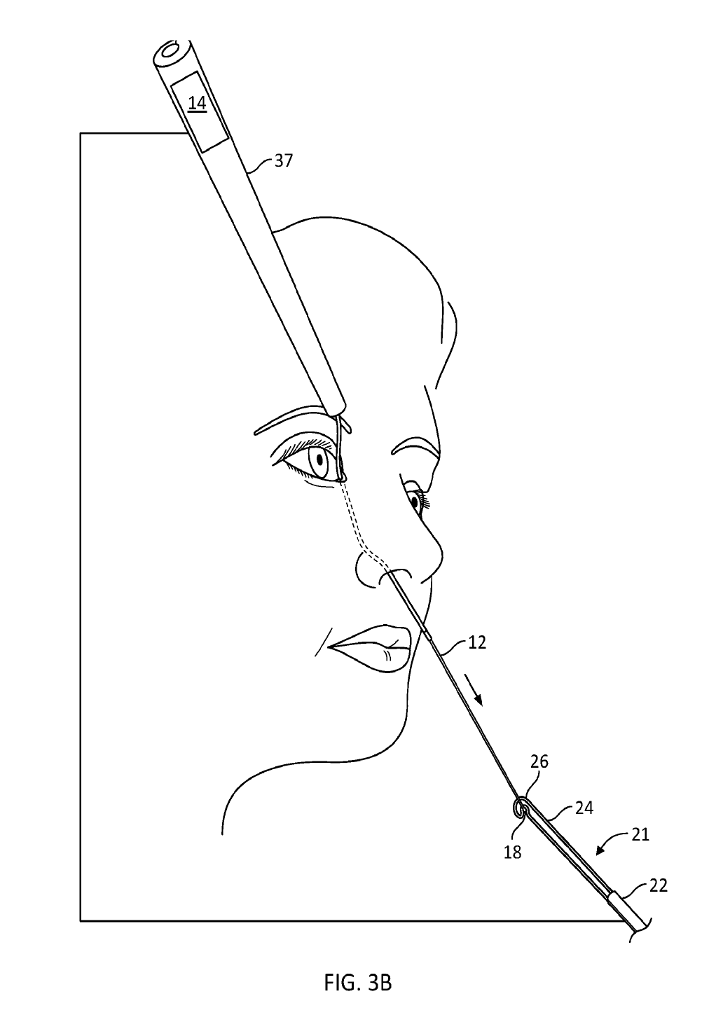

Although FIG. 1 shows various modules, in some implementations, two or more of these modules can be combined. For example, circuit 14 and/or signal generator 10 can be implemented in a miniaturized form (such as on an integrated circuit chip) and disposed on a portion of the probe or the tool. FIG. 3B illustrates an example of such a system where circuit 14 is disposed in or on a handle-shaped housing 37 attached to probe 12. In some implementations, housing 37 can include a switch 39 for switching circuit 14 on or off. In such cases, system 5 can be packaged, e.g., as a sterile package, as one self-contained system. The package can be sealed and sterilized using standard techniques such as radiation or chemicals, e.g., ethylene oxide gas, to form a kit. In some implementations, a portion of system 5 (e.g., probe 12 and circuit 14) can be packaged as a self-contained apparatus (e.g., using a housing 37) that can be used in conjunction with a commercially available tool 18. In some implementations, the self-contained apparatus can be provided as a sterile (and possibly disposable) unit that a clinician can use for an intubation procedure by simply opening the sealed package, removing the device using sterile procedures, and then performing the medical intubation without the need to connect any electrical leads to probes or other medical tools.

In some implementations, circuit 14 and signal generator 10 can be provided together in as a unit configured to accept an electrical connection from a probe 12 and tool 18 through appropriate leads. The leads can be made long enough to allow the unit to be sufficiently away from the sterile field of a surgical environment. The leads can be configured to be attached to probe 12 and tool 18 using electrical connectors such as an alligator clips or banana connectors.

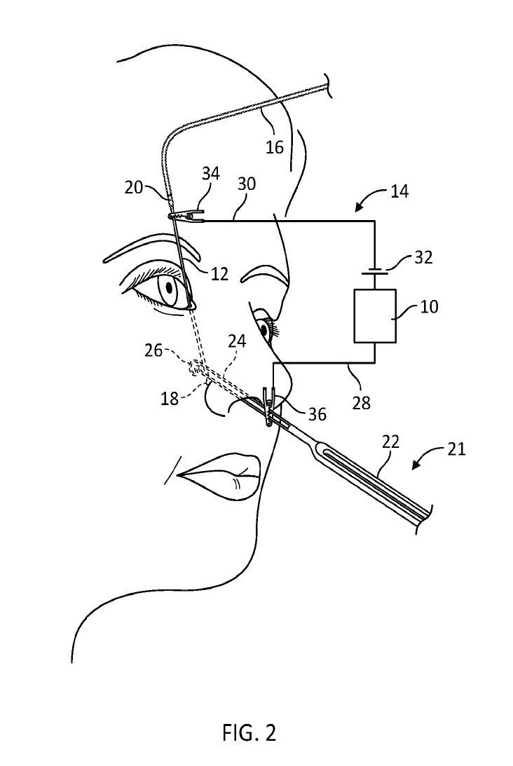

FIG. 2 illustrates parts of an example of a nasolacrimal intubation set (also known as a canaliculus intubation set). Although the complete set may comprise a pair of thin metal probes, only probe 12 is shown. In a complete set, probe 12 and a similar second probe can be fitted to respective ends of a tube 16 of silicone rubber which is limp and flexible.

Probe 12 can include an enlarged distal end portion or “olive” 18 and a tapered enlargement 20 adjacent to the proximal end of the probe 12. The end portion 18 is rounded and the enlargement 20 has an external size comparable to that of the tube 16. In some implementations, the probes are formed from a relatively fine tempered steel wire which is electrically conductive.

Probe 12 can move through the patient's lacrimal duct when pushed in a downward direction. The end portion 18 prevents the fine wire from puncturing the patient's tissue and is made, for example, from solder attached to the end of probe 12. Enlargement 20 can be formed in similar fashion. End portion 18 is generally spherical so that there is a limited possibility of damage caused by the end of the probe being forced to penetrate or scratch soft tissue.

Probe 12 can be used together with a tool 21 having a handle portion 22 with an aligned extension 24 terminating in a hook portion 26. The extension and hook portions 24, 26 are formed of stiff, electrically conductive wire and the hook includes a recess which extends forwardly and inwardly. The recess has a width corresponding generally to the diameter of the probe 12.

In use, probe 12 is pushed downwardly through the lacrimal duct and into the inferior meatus of the patient or subject. At this point, end portion 18 of the probe is in engagement with the lower wall of the meatus and the probe must be deflected to allow the end portion to be withdrawn. The probe can be deflected without undue force, which would otherwise result in damage to surrounding tissue, using the tool 21 to withdraw the end of probe 12 from the nasal passage, but the practitioner typically must first locate and “hook” the probe.

As indicated in FIG. 2, tool 21 is pushed into the nasal passage with extension 24 projecting inwardly. Because it is difficult or even impossible to look into the nasal passage, the usual practice is to feel around for the probe 12, and, once found to gently withdraw the tool to hook the end portion or “olive” 18 in the recess of hook portion 26. The tool is then withdrawn with end portion 18 trapped in the recess so that the tool can be used to draw the probe through the nasal passage and outwardly as illustrated in FIG. 3A.

This draws tube 16 into the lacrimal duct. The process can be repeated using the other probe (not shown) in the opposite canaliculus outflow system and after intubation of both systems the tube is cut leaving a portion in the canaliculus systems. The tube is then left in place for a period determined by the type of damage being corrected or repaired and then subsequently removed.

FIG. 2 shows a circuit that includes a pair of wires 28 and 30, connected to opposite contacts of a signal generator 10. Each wire has a free end connected to an alligator clip or other means 34 and 36 for attaching the wire to a conductive part of the probe 12 and tool 21. A battery or other power source or supply 32 sufficient to power signal generator 10, is provided at any appropriate location in circuit 14.

Signal generator 10 may, for example, be a low voltage buzzer, tone generator, LED (light-emitting-diode), vibrator, or other device that can generate an audible, visual, tactile, or other signal that can be perceived by a practitioner, when circuit 14 is closed to connect the power supply or source 32 to signal generator 10. Signal generator 10 may even generate a combination of two or more different types of signals, e.g., a tone and a rapidly blinking light, or a tone and a tactile signal, to advise the practitioner that he or she has made contact between the tool and the probe, even when the practitioner is not directly looking at the signal generator 10, or when there is other noise in the operating location.

Power source 32 may be a battery holder for receiving a battery, within the meaning of this disclosure since the battery will normally be a consumable and replaceable part used with the apparatus. The compact size and simple and cheap construction of circuit 14 makes it readily available to practitioners in any country.

Although attachable means in the form of clips 34 and 36 are illustrated, the circuit can be incorporated into tool 21, which may contain signal generator 10 and battery 32 along with a single wire or lead 30 and clip 34 for engaging a conductive part of probe 12, which is still outside the patient.

In addition, attachment means other than clips and of any known type can be used for connecting the circuit 14 to conductive portions of the tool and probe. Contact between tool extension 24 and probe 12 amounts to closing a simple switch for circuit 14, but this simple action gives the practitioner a quick and easy way of finding the end of probe 12.

Disclosed herein are improved circuits that replace the circuits found in the prior art and allow for low power and low current implementation. Such a low power device can be used for fast positioning of a tube into the lacrimal duct. The new circuits can be configured to limit the current (and resulting power dissipation) through the probe below a predetermined threshold. Such limiting of the current in the probe allows for implementation of the new devices that can be safely used in human subjects without causing pain, discomfort, or other adverse conditions that can result from high current flow through sensitive tissues.

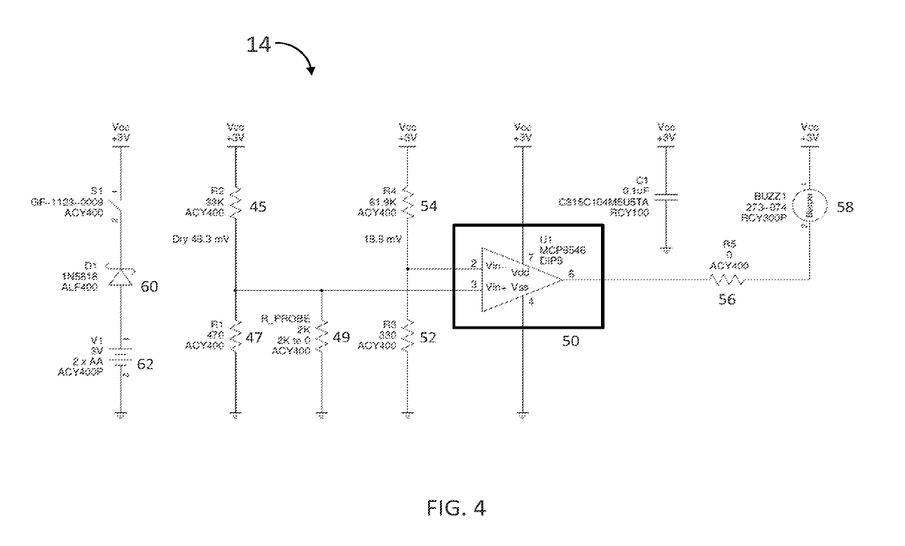

FIG. 4 is a circuit diagram illustrating of an example of a circuit 14. These circuits are current limited, voltage limited, and/or resistant to a reasonable electrostatic discharge levels. In some implementations, circuit 14 uses a voltage divider to detect continuity between two connected probes or a probe and a tool. For safety, the current and/or voltage on the probes can be limited. In some implementations, safety and stability can be further improved by battery isolation. In some implementations, a voltage divider configuration can be used in circuit 14 for limiting the current through the probe.

In some implementations, circuit 14 can include two voltage divider configurations. The first voltage divider can include three resistors 45, 47, and 39 with values R1, R2, and R_Probe, respectively. To limit the worst case current through resistor 49, the value R2 can be selected assuming the value R_Probe to be zero. Such an assumption increases a likelihood of preventing a current flow through resistor 49 in the presence of any tissue was between the probe and the tool. The circuit shown in FIG. 4 can be designed, for example, using the following design parameters:
a) Maximum current in probes R_Probe_Imax=200 μA
b) Typical current in the probes: about half the maximum value, i.e. R_Probe_Ityp=R_Probe_Imax/2
c) Soft moist tissue conductivity: R_Probe=2KΩ
d) System voltage: V1=Vcc=3V

The value resistor 45 is therefore given as:

$$\begin{matrix}R 2 = V 1 / R\_Probe \_Ityp \\ = 30 K Ω\end{matrix}$$

In some implementations, a standard resistor value, such as 33KΩ can be chosen for resistor 45.

In some implementations, resistor 47 functions as a shunt resistor such that a majority of the current passes through resistor 47 and not the probe and the tool. Under such conditions, a low resistance value, such as 330Ω or 470Ω can be chosen as a value R1 for resistor 47. When a current path through the probe is closed, resistor 49 (or the resistance value R_probe) represents the current path. The current through resistor 45 is given by:

R2_{—}Idry=V1/(R1+R2)

The voltage across the probe and the tool (when dry) is given by:

R_Probe_{—}Vdry=V1_{—}R2_{—}Idry*R2

The current through the probe when the probe is wet is given as:

R_Probe_{—}Iwet=V1/(R_Probe_{—}thRwet+R2)

wherein

R_Probe_{—}thRwet=(R1*R_Probe)/(R1+R_Probe)

The voltage across the probe and the tool, when the probe is wet, is given by:

R_Probe_{—}Vwet=R_Probe_{—}Iwet*R_Probe_{—}thRwet

When the probes are touching, the current through the probe is given by:

R_Probe_{—}Itouch=V1/R2

In some implementations, the circuit includes a comparison module 50, which can include, for example, a comparator such as the U1 MCP6546 DIP8 comparator shown in FIG. 4. In some cases, the comparator can be chosen such that it has a sufficiently wide hysteresis loop that prevents the comparator from self oscillating. In some implementations, it may also be of interest to choose a relatively slow comparator to prevent the self oscillations. Threshold conditions for hysteresis and speed can be used in choosing the comparator. In some implementations, a comparison module 50 with a response time of 7 or more can be selected. In some implementations, the reference voltage of the comparison module is such that the reference voltage is less than the voltage when the probes are wet and more than the voltage when they are touching.

The voltage across the probe and the tool can be selected, for example, to be a multiple of an input offset voltage of comparator 50. The ratio between the value R2 and the parallel combination of the values R1 and R_Probe can be used to determine the voltage across the probe and the tool. In some implementations, the difference between the voltage across resistor 49 and the reference voltage is determined to be about half way between the sense voltage (i.e., the voltage at the probe) and zero such that comparison module 50 can detect a difference that is larger than a threshold amount. This can ensure that the comparison module 50 does not detect small differences that could potentially render the comparison module unstable. For example, the reference voltage V_Ref can be given by:

V_Ref=R_Probe_{—}Vwet/2

In some implementations, a second voltage divider configuration can be used to provide the reference instead of a voltage regulated source. Therefore, the reference voltage levels can be made to change substantially proportionally with the voltage across resistor 49 when the electrodes are not touching. By allowing the relative voltages to be maintained while the rail voltage changes circuit 14 can be configured to be powered using unregulated battery power. In some implementations, this can reduce component count in circuit 14 and/or increase battery life. The second voltage divider configuration can be implemented, for example using resistors 52 and 54 with values R3 and R4, respectively. Resistors 52 and 54 can be chosen such that even when two metal tools or probes touch each other the current shunted will be lower than a predetermined threshold. In some implementations, the value R4 can be chosen as:

R4=R2*2

In some cases, resistor 54 can be chosen to have values such as 66 KΩ or 61.9 KΩ. The current through resistor 54 is given by:

R4_{—}I=(V1−V_Ref)/R4

The value R3 for resistor 52 can be chosen as, for example:

R3=V_Ref/R4_{—}I

In some implementations, the response time of comparison module 50 is selected to be more than a predetermined threshold, e.g., 7 μs. The slow response time can help improve the stability of comparison module 50 by reducing the possibility of high frequency oscillation. This in turn can also improve precision of the comparison module. In some implementations, the comparison module can include a comparator with internal electrostatic discharge protection. This reduces the need for a separate electrostatic discharge protection circuit, thereby reducing the component count of the circuit. For example, internal electrostatic discharge protection for comparison module 50 can be provided by using a comparator that has an open collector output with a sufficiently large current rating, e.g. 30 mA. In some implementations, using a buzzer with an internal oscillator further reduces the component count for circuit 14.

In some implementations where the exact calculated values for the resistors are not available, approximate or substantially close available values can be used. In some implementations, resistor 56 can be used to set a gain for signal generator 58 (e.g., a volume of a buzzer or beeper). The value R5 of resistor 56 can be set experimentally. In some implementations, other alarms or alerting modules can be used in place of, or in conjunction with, the buzzer as the signal generator. For example, the circuit can include a display that visually renders establishment of continuity. The circuit can also include a mechanism for providing haptic or tactile feedback in place of or in conjunction with the buzzer. In some implementations, a light emitting diode (LED) can be used as the signal generator. In such cases, a diode such as a Zener diode can be used to adjust the light emission from the LED.

The source of power for the voltage rails (Vcc) can be, for example, a battery 62 that includes, for example, two 1.5V AA cells for a total of 3.0 volts. In some implementations, electricity from a building electric system can also be used as the source of power. However, the use of a battery can provide isolation from utility power lines as an additional safety factor. In some implementations, circuit 14 can be housed in a box made from non-conductive material. In some implementations, the circuit includes another diode, such as the Schottky diode 60 to prevent the circuit from malfunctioning if the batteries are installed backwards.

The circuits described herein can be implemented in various ways. For example, the circuits can be implemented on a printed circuit board. In some implementations, the circuits can include one or more integrated circuits. The circuits described herein can be implemented using more or fewer circuit elements than what is depicted in FIG. 4.

Circuit 14 is designed such that the current or power through the probes do not exceed a threshold condition. The threshold condition can be chosen based on a level of current or power that can be tolerated by the corresponding human body part. For example, the circuit can be designed such that the current does not exceed 200 μA. The threshold conditions can be determined in various ways, for example, by experimentally or theoretically determining a resistance of a specific portion of the human body.

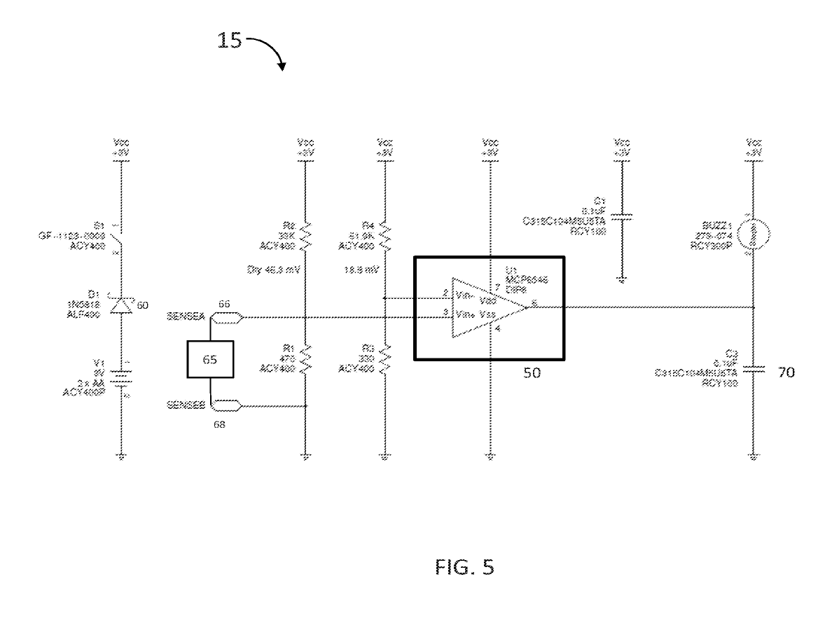

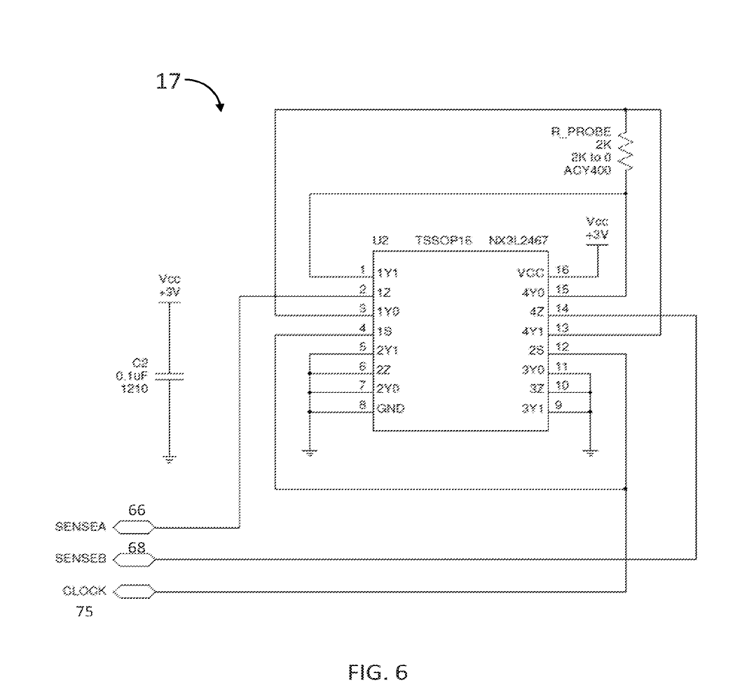

FIG. 5 illustrates an example of another circuit, circuit 15, which can be used as an alternative to circuit 14. Circuit 15 includes a double pole double throw (DPDT) switch 65 between the probes (or the probe and the tool) 66 and 68. Switch 65 can be electronically controllable and can be automatically switched at an even duty by an oscillator with a predetermined periodicity. FIG. 6 illustrates a device 17 that can be used to implement switch 65. In some implementations, device 17 can be a solid state device with internal electrostatic discharge protection. The frequency of oscillation can be selected to be non-interfering with frequency used for utility power transmission (e.g., 60 Hz, 50 Hz, or 400 Hz). Switch 65 can be implemented by configuring the device 17 to reverse the connections to the probes 66 and 68 every time the clock 70 cycles. In some implementations, the switching speed of DPDT switch 65 can be selected to be faster than the response time of comparison module 50. This can mean that the opening of switch 65 between states does not change the output of comparison module 50. In some implementations and as shown in FIG. 5, a capacitor 70 can be added to introduce additional hysteresis that also further reduces the response time of comparison module 50.

### Other Embodiments

The methods and systems described herein can also be adapted to other medical procedures utilizing probes extending through body passages where the need exists for locating one end of the probe. While some specific embodiments have been described in detail, other embodiments, aspects, advantages, and modifications are within the scope of the following claims.

1. An apparatus comprising:
an electrically conductive probe configured to be extendible through a body passage
a conductive tool for locating the probe in the body passage;
a signal generator configured to generate a feedback signal;
a circuit coupled to the probe and the conductive tool, wherein the circuit comprises a comparison module that is configured to compare a voltage developed at the probe with a reference voltage and cause a generation of the feedback signal if at least a portion of the conductive tool touches the probe, where the circuit is further configured to prevent the generation of the feedback signal when no portion of the conductive tool touches the probe; and
a power source for powering the signaling mechanism to generate the signal,
wherein the circuit is configured such that a current through the probe is below a first predetermined threshold.

2. The apparatus of claim 1, wherein the signal generator is selected from a group consisting of a visual signal generator, an audible signal generator, and a tactile signal generator.

3. The apparatus of claim 1, wherein at least a portion of the circuit is disposed on the conductive tool.

4. The apparatus of claim 1, wherein at least a portion of the circuit is disposed on a housing coupled to the probe.

5. The apparatus of claim 1, wherein a response time of the comparison module is greater than a second predetermined threshold.

6. The apparatus of claim 5, wherein the second predetermined threshold is 7 μs.

7. The apparatus of claim 5, wherein the second predetermined threshold is between 2 μs and 7 μs.

8. The apparatus of claim 1, further comprising a shunt resistor connected in parallel to the probe and the conductive tool, wherein the shunt resistor is configured to limit current through the probe below the first predetermined threshold.

9. The apparatus of claim 1, wherein the comparison module comprises a comparator that is configured to withstand electrostatic discharge up to a third predetermined threshold value.

10. An apparatus comprising:
an electrically conductive probe configured to be extendible through a body passage a signal generator configured to generate a feedback signal; and
a circuit coupled to the probe, wherein the circuit comprises a comparison module that is configured to compare a voltage developed at the probe with a reference voltage and cause a generation of the feedback signal if at least a portion of the probe touches a conductive tool used to locate the probe in the body passage, wherein the circuit is further configured to prevent the generation of the feedback signal when no portion of the conductive tool touches the probe,
wherein the circuit is configured such that a current through the probe is below a first predetermined threshold.

11. The apparatus of claim 10, wherein the signal generator is selected from a group consisting of a visual signal generator, an audible signal generator and a tactile signal generator.

12. The apparatus of claim 10, wherein at least a portion of the circuit is disposed on a housing coupled to the probe.

13. The apparatus of claim 10, wherein a response time of the comparison module is greater than a second predetermined threshold.

14. The apparatus of claim 13, wherein the second predetermined threshold is 7 μs.

15. The apparatus of claim 13, wherein the second predetermined threshold is between 2 μs and 7 μs.

16. The apparatus of claim 10, further comprising a shunt resistor configured to limit the current through the probe below the first predetermined threshold.

17. The apparatus of claim 10, wherein the comparison module comprises a comparator that is configured to withstand electrostatic discharge up to a third predetermined threshold value.

18. An apparatus comprising:
a signal generator configured to generate a feedback signal; and
a circuit configured to be coupled to an electrically conductive probe that is extendible through a body passage and a conductive tool for locating the probe within the body passage, the circuit comprising:
a comparison module that is configured to compare a voltage developed at the probe with a reference voltage and to cause a generation of the feedback signal if at least a portion of the probe touches the conductive tool, wherein the circuit is further configured to prevent generation of the feedback signal when no portion of the conductive tool touches the probe, and wherein the circuit is configured such that a current through the probe is below a first predetermined threshold.

19. The apparatus of claim 18, wherein the signal generator is selected from a group consisting of a visual signal generator, an audible signal generator and a tactile signal generator.

20. The apparatus of claim 18, wherein a response time of the comparison module is greater than a second predetermined threshold.

21. The apparatus of claim 20, wherein the second predetermined threshold is 7 μs.

22. The apparatus of claim 20, wherein the second predetermined threshold is between 2 μs and 7 μs.

23. The apparatus of claim 18, further comprising a shunt resistor configured to limit the current through the probe below the first predetermined threshold.

24. The apparatus of any claim 18, wherein the comparison module comprises a comparator that is configured to withstand electrostatic discharge up to a third predetermined threshold value.

25. The apparatus of claim 18, further comprising connectors to electrically couple the circuit with the probe and the conductive tool.

26. A method comprising:
providing a circuit configured to be coupled to an electrically conductive probe that is extendible through a body passage, and a conductive tool for locating the probe within the body passage, the circuit comprising a comparison module that is configured to compare a voltage developed at the probe with a reference voltage and cause a generation of a feedback signal, wherein the circuit is further configured to prevent the generation of the feedback signal when no portion of the conductive tool touches the probe, and wherein the circuit is further configured such that a current through the probe is below a first predetermined threshold; and
locating, based on the feedback signal, the probe when at least a portion of the probe touches the conductive tool.

27. The method of claim 26, wherein the body passage is a nasolacrimal duct.

28. (canceled)
